(19) United States
(12) Patent Application Publication
(10) Pub. No.: US 2011/0046639 A1
(43) Pub. Date: Feb. 24, 2011

(54) Method of direct hair implantation

(76) Inventor: Konstantinos Giotis, Athens (GR)

Correspondence Address: GIOTIS KONSTANTINOS, 30, VOULIAGENIS AVENUE, NEOS KOSMOS, 117 43 ATHENS, GR

(21) Appl. No.: 12/461,745

(22) Filed: Aug. 24, 2009

(51) Int. Cl. A61B 17/50; A61B 17/34

(52) U.S. Cl. 606/133; 606/187

(57) ABSTRACT

Method of direct hair implantation consisting in sequentially harvesting single hair follicles from a donor region and directly implanting the same at a recipient region of the scalp, wherein the method employs a hair harvesting instrument with a sterile disposable punch of appropriately small diameter adapted to successively perform cutting of a single hair follicle when it is brought in contact with the root of the hair follicle and is manually rotated around the perimeter of the same. A micro gripper means is thereafter used for the extraction of the cut hair follicle and for loading a forwardly projecting tubular needle of a hair implanting instrument with the extracted hair follicle. A hair implanting instrument is then employed for a selective placement of said hair follicle in the appropriate direction and at an appropriate position on a demarcated recipient region of the scalp. The process of single hair follicle harvesting, extraction and direct implantation is repeated until completion of the planned direct hair implantation procedure.

### THE FIELD OF THE ART

The invention relates to a direct hair implantation method and in this respect it discloses appropriate transplanting equipment enabling sequential harvesting of single hair follicles from a donor region and implantation of the same at a recipient zone of the scalp.

### THE BACKGROUND OF THE INVENTION

Various hair implantation procedures and equipment associated with the implementation thereof have been employed in the prior art. The transplantation of hair grafts into a human scalp is well understood and has for a long time been practiced by means of the strip surgery hair transplanting technique. This prior art method of hair transplantation relates to excising a donor hair-rich area of the scalp, dissecting the excised scalp segment to obtain individual hair grafts for implantation, creating a recipient implantation site in a bald or thinning area of the scalp by making wounds with a cutting instrument, and then implanting individual grafts into the prepared implantation sites after bleeding has ceased.

As hair transplantation market developed, the Follicular Unit Extraction (F.U.E.) method of hair transplantation was introduced that limited the transplanting process to creating follicular units that contain one to four hairs bunched together as they are found in a normal human scalp and using these follicular units as hair grafts. Appropriate instruments were accordingly provided to surgeons to implant hair grafts, such instruments having been designed for placing hair grafts into pre-made wounds. A typical, commercially available hair graft implantation instrument includes a hollow needle within a sheath that is alternately moved between an extended position in which the needle extends beyond the sheath and a retracted position in which the needle is retracted within the sheath. In another class of instruments, a single instrument makes a scalp wound and implants a hair graft into the freshly made wound in a rapid sequence of mechanical actions.

By way of example, WO-2005/109799 discloses a follicular extraction method and device, whilst U.S. Pat. No. 6,059,807 presents a device and method for implanting small-sized grafts (approximately 1 mm in diameter and 5 mm in length), called micro grafts, that are generally gripped by means of micro grippers and then manually reimplanted, one by one, in respective receiving sites prepared beforehand in the scalp.

The abovementioned hair transplanting process of the prior art presents a number of drawbacks. Strip removal necessitates shaving and leaves a scar that is difficult to heal, thereby posing restrictions in the client's normal activities over a rather long healing period. The process is rather slow with short daily sessions enabling implantation of a limited number of hair of the order of 500 units, whilst its efficiency is rather low due to a large percentage of up to 20% of follicles being damaged during the removal of the strip and the subsequent division thereof in individual follicles to be implanted into the recipient zone. Eventually since a number of sessions must take place to perform the overall hair transplantation process and since such sessions have to be scheduled with extensively long intervals in between, it becomes uncomfortably long to complete the process. Furthermore, placement of hair follicles is subsequently performed by means of forceps thereby leaving much room for human error that often leads to oddly directioned hair at incorrect angles instead of the desired natural result of evenly angled hair. Moreover such graft extraction and subsequent implanting process is not available for all clients, e.g. very curly hair cannot be extracted, and a preliminary test has to be conducted to determine whether the process is suitable for each particular candidate.

WO-2009/083741 disclosed a hair transplanting device and method for the use thereof. The method disclosed in WO-2009/083741 relates to sequentially harvesting single hair follicles from a donor region and implanting the same at a recipient region of the scalp. This single hair transplanting process is an advanced method since it eliminates strip surgery and adverse effects encountered with the same.

Moreover the above innovative single hair transplanting process has been made possible through employment of newly disclosed specialized tools, including a hair implanting device comprising a hollow needle having a longitudinal groove at a frontal portion thereof with an obliquely cut free end adapted to receive a follicular hair unit, a sliding rod being reciprocatingly movable axially along the needle to effect placement of the follicular hair unit disposed within the groove of the frontal end of the needle within a predetermined position of the scalp at the recipient bald area, and a tubular housing adapted to receive said needle and said sliding rod incorporating means of performing the hair implanting operation. A particular advantage of this hair implanting device was the provision of means of appropriately regulating and fine adjustment of the depth of intrusion of the needle into the scalp to provide for optimum performance of the hair implanting process. Other implanting devices, such as the one disclosed in the method for hair transplantation disclosed in U.S. Pat. No. 5,578,054 comprised an instrument having a shaft and a blade that is attached to the shaft normally to the axis thereof and is inserted into the skin to a preselected depth as determined by a stop disposed along the shaft to form an incision, whereby a graft of skin having at least one hair is placed in the incision following removal of the instrument.

However, again this method of minigraft (2 to 6 number of hair) or even single hair transplanting, although substantially advantageous in comparison to strip surgery operations of the prior art, still necessitates preparing a recipient site in the scalp for hair implanting and performing the implantation process after having extracted hair follicles from the donor region and after having treated and processed the same under the microscope. As a consequence of this hair transplanting approach comprising two discrete main steps of hair extraction and hair implanting, hair has to remain a rather extensive period out of the body and this necessitates treatment thereof with normal saline in Petri dishes, yet without completely eliminating damages caused through their handling and possible dehydration. Such possible damages might be made more apparent if the fact of hair handling by assistant staff of relatively low skills undertaking to implement the process of processing the grafts under the microscope, placing the same in Petri dishes, counting and eventually loading them in the implanting devices, is taken into consideration.

Furthermore a very important factor determining successful implementation of a hair transplanting process is the fatigue of both the patient, who has to lie for long periods of time in a rather uncomfortable position with his head facing downwardly, and of the medical team performing the process. In relation to the latter, it is obvious that the substantially advanced and precise technique of single hair transplanting necessitates a continuous attentive effort from the surgical team for many hours and it is well known that long procedures make the people involved prone to mistakes, whilst the rate of survival of hair grafts is substantially reduced in proportion to the duration of the procedure. Finally, considerable doses of anesthesia and of postoperative medication are still necessary also in this single hair transplanting process. The aforementioned disadvantages of the hair implanting process of the prior art necessitating preparation of a recipient site in the scalp for hair implanting and performing the implantation process after having extracted hair follicles from the donor region and after having treated and processed the same under the microscope are encountered in a variety of hair transplanting devices and procedures, such as in U.S. Pat. No. 6,059,807 or U.S. Pat. No. 7,452,367.

It is therefore the object of the present invention to advantageously overcome and eliminate the abovementioned disadvantages and drawbacks of the prior art. With this scope in mind, the invention proposes a minimally invasive, substantially non-traumatic, much faster and much more comfortable direct hair implanting process that is available to all potential candidates without exception and requires neither shaving of the scalp nor removal of grafts or any preliminary testing whatsoever. In accordance to this direct hair implanting process single hair follicles are extracted from a donor region and are subsequently immediately implanted at a recipient region. The process is fast providing the possibility of extracting and directly implanting an approximate number of 2,000 hair units in one session.

It is a further object of the invention to provide the aforementioned direct hair implantation technique in association with appropriately, ergonomically designed equipment and furniture making possible implementation of the overall process with both the surgeon and the client comfortably seated, thereby minimizing the fatigue factor associated with the rather long lasting session requirements and further provide for the much faster conclusion of the process with substantially reduced anesthesia and postoperative medication, the shortening of the duration of the operation being due to the elimination of the step of previously preparing the hair recipient site in the scalp and appropriately waiting for the recipient site to stop bleeding in order to initiate the implanting process and to elimination of the intermediate step of hair handling and processing in between the initial step of hair extraction and the eventual step of hair implanting. Further, the less invasive and less traumatic property of the herein proposed direct hair implanting operation and the significantly reduced bleeding during the procedure and reduced scarring after conclusion thereof is associated with the employment of punches of smaller diameter, ideally of the order of 0.7-0.85 mm.

### BRIEF DESCRIPTION OF THE DRAWINGS

The invention will be made apparent to those skilled in the art by reference to the accompanying drawings in which:

FIG. 1 shows a typical hair follicle cutting instrument employed in the invention.

FIG. 2 shows a typical hair implanting instrument employed in the invention and FIG. 2a shows a detail of the frontal needle portion of the hair implanting instrument of FIG. 2.

FIG. 3 shows application of the hair follicle cutting instrument shown in FIG. 1 during a hair follicle harvesting operation.

FIG. 4 shows a micro gripper employed in an extraction operation of the hair follicle previously cut in the hair follicle harvesting operation depicted in FIG. 3.

FIG. 5 shows a hair follicle loading operation of the hair implanting instrument shown in FIG. 2.

FIG. 6 shows two consecutive steps in a hair implanting procedure with the hair implanting instrument operated by hand.

FIG. 7 shows two consecutive steps in a hair implanting procedure with a magnified cross sectional view of the scalp at the recipient area.

### DETAILED DESCRIPTION OF PREFERRED EMBODIMENTS

The aim of the technique disclosed in the present invention is the extraction of hair follicles from the back of the scalp, from the occipital and temporal area that is known to be the area of permanent hair, hereinafter being referred to as the donor area and the immediate placement of those hair follicles to the area of temporary hair that is known to be the area of the forehead, middle and crown area of the scalp, hereinafter being referred to as the recipient area, in order to restore the permanent hair loss that has occurred in this area due to various reasons, most commonly due to androgenetic alopecia.

The direct hair implanting process is a minimally invasive technique that takes place directly, without the interference of Petri dishes or graft processing under the microscope, with the patient on seated position. The proposed process ensures significantly less bleeding, trauma and pain, both in the donor and the recipient area. In addition, the direct extraction and placement of hair follicles, one by one, constitutes the core of the proposed method and contributes essentially in a positive manner to the improvement of hair follicle survival and overall hair growth, as handling of the hair follicles is minimal and the time the hair follicles remain out of the body is practically eliminated.

The procedure is carried out by a team of experts that may consist of medical practitioners, technicians and nurses, properly trained and certified as direct hair implanting medical specialists.

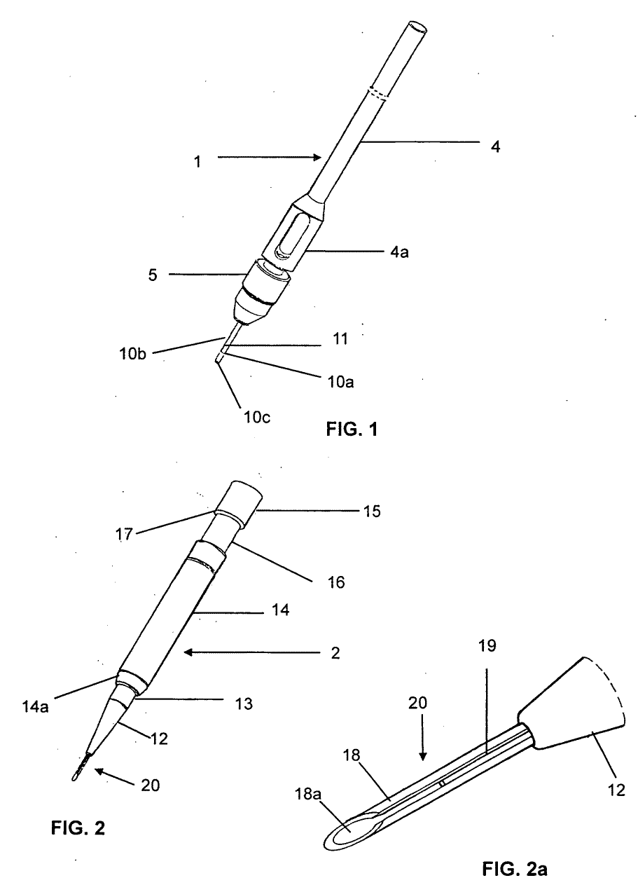

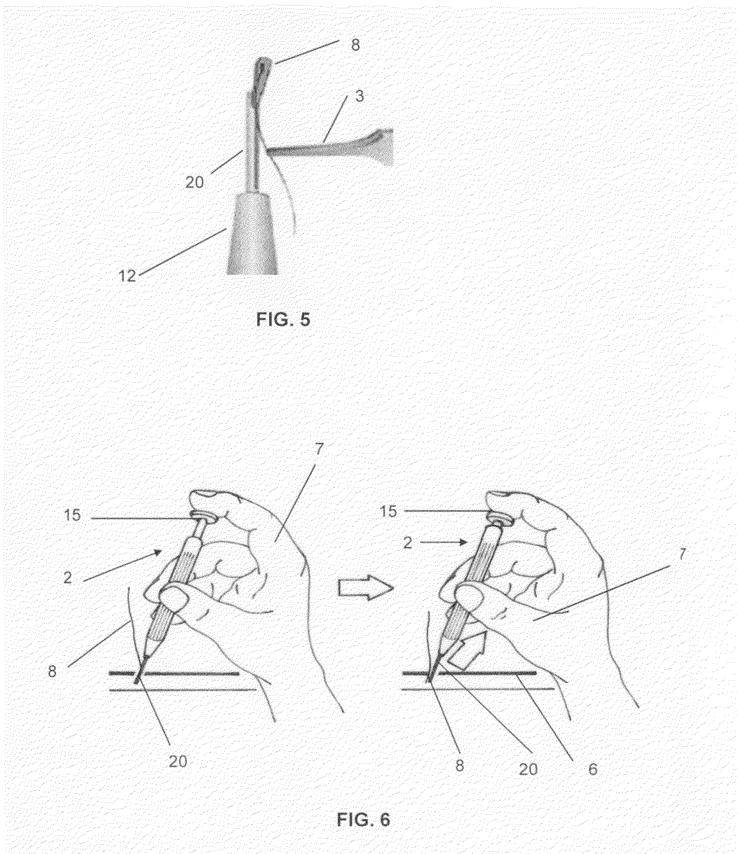

The tools employed in the direct hair transplanting process of the invention is a hair harvesting device 1 depicted in FIG. 1, a hair implanting device 2 depicted in FIG. 2 and a micro gripper 3 that is used to extract the hair follicle previously cut by means of the hair harvesting device 1 (FIG. 4) and subsequently load the hair implanting device 2 (FIG. 5) with the hair follicle ready to be implanted into a recipient site of the scalp.

The hair harvesting device 1 of FIG. 1 comprises a tubular cutting head that is divided along a circumferential line 11 in a frontal part 10a and a rear part 10b, wherein the frontal part 10a is provided with an appropriate coating, gold or equivalent, to provide for a medically acceptable contact with the human body. Whilst a handle means for the tubular cutting head is not necessary, a handle means is preferably used to facilitate the process and, if this is the case, the rear part 10b of the tubular cutting head is appropriately connected at the frontal end of the handle means. The handle means is a cylindrical shank 4 that extends at a connecting extension member 4a that is introduced within a through axial opening of a choke means 5 provided with a frontal jaw that fixedly supports the hair harvesting tubular cutting head. The frontal part 10a of the tubular cutting head is formed with a conical knife edge cutting surface 10c adapted to successively perform cutting of single hair follicles when it is brought in contact with the root of the hair follicle and is manually rotated around the perimeter of the same. The diameter of the knife edge cutting surface 10c of the tubular cutting head 10 of the invention may vary within a range of 0.40-2.00 mm to provide for hair harvesting operations in the overall hairy geography of the human body. However with a scope of significantly reducing bleeding during the hair harvesting procedure and equivalently reducing scarring after conclusion thereof the punches employed are of a smaller diameter, ideally of the order of 0.4-0.8 mm.

The hair implanting device 2 shown in FIG. 2 comprises a longitudinal hollow needle 20 with a holder means inserted within a frontal conical supporting portion 12 of the hair implanting instrument and a push rod 19 that extends all along the tubular housing 14 of the implanting instrument and is provided with a holder means that is located within a first portion 16 of the rear push button assembly of the device. As shown in FIG. 2a, needle 20 is provided with a longitudinal groove 18 being formed at the frontal portion thereof that projects outwardly from the conical supporting portion 12 with an obliquely cut free end 18a, wherein longitudinal groove 18 is adapted to receive a follicular hair unit and wherein push rod 19 is reciprocatingly movable axially along the interior of needle 20 to effect placement of the follicular hair unit disposed within the groove 18 of the frontal end of the needle at a selected position of the scalp at the recipient bald area. It is hereby noted that the obliquely cut free end 18a of needle 20 is sharpened so as to effect direct implantation of the hair follicle loaded therein without prior incision of the recipient site of the scalp and so that in accordance with an alternative preferred embodiment of the invention the same implanting instrument 2 might also be employed in a hair follicle cutting operation also.

The hair implanting device 2 comprises a tubular housing 14 that incorporates a plurality of various parts adapted to receive needle 20 and sliding push rod 19 and means of preferentially regulating and fine adjustment of the depth of intrusion of the needle into the scalp to provide for optimum performance of the hair implanting device. The obliquely cut free end 18a of needle 20 comprises inwardly cut sharpened surfaces so that it might be, capable of performing an incision at the recipient area of the scalp suitable for receiving the hair follicle to be implanted therein, such incision being made simultaneously during carrying out the hair follicle implantation process. Since needle 20 is fixedly mounted within the centrally located tubular housing 14, a further plastic cylindrical item is employed the frontal conical end 14a of which extends forwardly of the tubular housing 14, such item being provided with threading screwable around a correspondingly threaded portion of tubular housing 14. Movement of this plastic item through differentiated screwing thereof along the stroke determined by the length of aforementioned mutual threading results in a corresponding differentiated protrusion of needle 20 through the frontal outlet of the conically formed frontal supporting portion 12.

Following assembly of the needle and push rod assembly within tubular housing 14 of the hair implanting instrument, a longitudinal plastic housing 13 that forwardly extends at the frontal conical supporting portion 12 is guided within the tubular housing 14 to integrate the entire assembly in combination with a rear push button assembly. The longitudinal plastic housing 13 is a hollow cylindrical unit with the frontal conical supporting portion 12 and a rear portion that is fixedly connected to the rear push button assembly.

The rear push button assembly is a plastic hollow cylindrical housing with a first portion 16 and a second portion 15, wherein second portion 15 has a diameter marginally larger than the diameter of the first portion 16 so as to externally form a circumferential basement 17 that acts as a terminal means of the push button operation when it touches onto the rim of the rear open end of the tubular housing 14 whilst the first portion 16 thereof has been inserted within the latter.

The push button means 15, 16 provided at the rear of the hair implanting instrument is used for initiating a forward stroke of push rod 19 slidable within the interior of needle 20. During a hair implanting operation a forward stroke of push rod 19 results in effecting unloading into the recipient locus of the scalp of the hair follicle loaded within the concave frontal cavity 18 of needle 20, whilst a spring means incorporated axially along push rod 19 within the tubular housing 14 of the device is being compressed during such forward stroke and when, following conclusion of a hair implanting operation, push button means 15, 16 is released, the aforementioned spring means acts so as to activate a rearward movement of push rod 19 back to rest position, ready for a new hair follicle loading operation.

A first step, prior to the application of the hair implantation procedure of the invention, is the review of the overall medical history of the client along with the results of a diagnostic Alopecia Test, already completed in a previous visit.

The procedure is thereafter being planned with the medical specialist proceeding with planning of the hair line, the design and distribution of the hair follicles to be transplanted. A surgical pen is used to demarcate the limits of the recipient area and their distribution and preoperative photos are taken.

The assigned technician proceeds with disinfection of the hair by washing with an antiseptic shampoo (e.g. povidone iodine shampoo). Then, the technician cuts the hair to the desired length, according to the amount of hair agreed before the procedure. There are three options of hair cut that accordingly effect the duration of the hair implanting procedure:

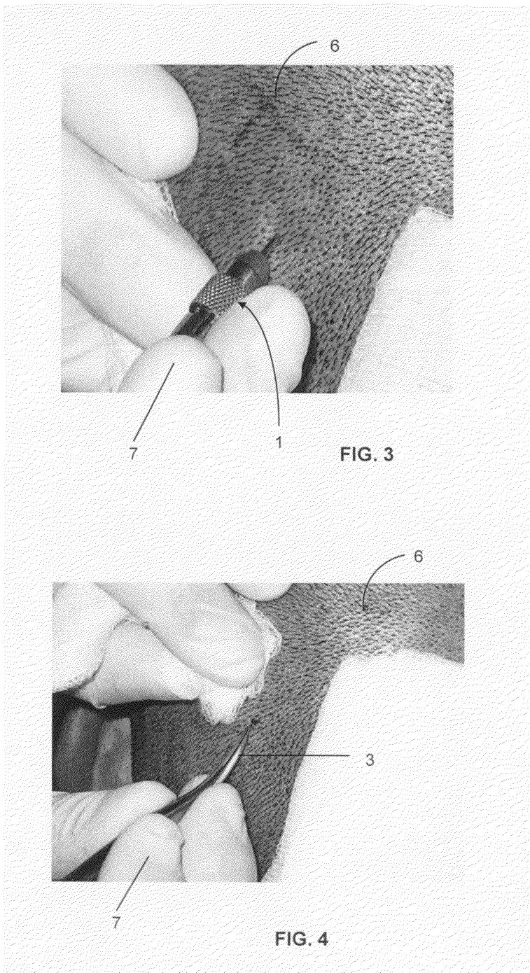

A first option is shaving of hair up to 0.5 cm length that allows for optimum harvesting of the donor area. This option is selected in the case of the scalp 6 depicted in FIGS. 3 and 4.

An alternative option is line shaving of one or two zones, reserved for women or men with long hair who would not want to shave their hair. Harvesting of hair follicles if this mode of line shaving is employed may be limited.

Yet another option is totally unshaven hair, whereby the operation is performed without prior shaving, the total amount of harvested grafts is smaller and the extraction may take longer to be completed.

It is also optional to apply, during hair cutting, if necessary, a dark dye onto white hair in order to make the hair roots visible and facilitate the extraction of hair follicles.

Following the above preparatory phase, the hair follicle harvesting process can take place with the patient seated on a properly adjustable seat with adjustable neck support. The position of the patient is carefully selected by the medical practitioner in charge and determined in accordance with each specific hair restoration procedure being planned.

The limits of the donor area are then demarcated in the occipital and temporal areas with the surgical pen.

An antiseptic solution (e.g. povidone iodine 10%) is then applied on the donor area for at least 1 min, in order to disinfect the skin. After disinfection, sterile natural saline is used to remove the disinfectant. Although this is a minimally invasive technique and the administered dosage of anesthesia is low, the procedure is rendered yet less invasive if in accordance with a preferred embodiment of the invention, a local anesthetic cream or gel (e.g. EMLA cream, Ametop gel) is applied in combination with ice.

The hair restoration procedure starts with the administration of a prepared solution of local anesthesia containing lidocaine 1% and adrenaline 1:200000. The preparation of the solution is carried out in accordance with a predetermined protocol of local anesthesia administration.

In accordance with a preferred, illustrative embodiment of the invention, local anesthesia is administered intradermally with a 30G needle in the donor area in a ring block manner. Administration starts at the lower edge of the donor area and continues around the whole donor area to complete the ring block. Thus, effective, long lasting local anesthesia and vasoconstriction is achieved with the smallest possible dosage of local anesthetic administered.

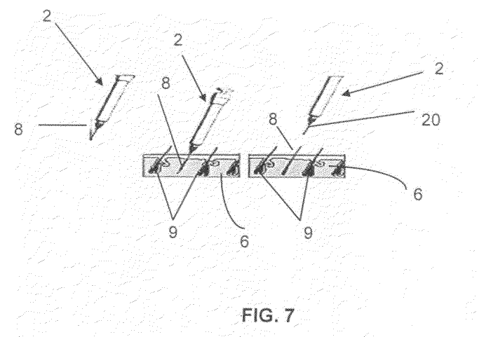

After satisfying anesthesia is achieved, cutting of the hair follicle starts using a sterile disposable punch preferably of appropriately small diameter within the range of 0.4 mm-0.8 mm. A typical punch employed is shown in FIG. 1, such punch comprising a cutting head mounted at the choke of a shank adapted to perform hair harvesting operations, whilst employment of such hair harvesting instrument 1 onto the scalp 6 of a client by the medical practitioner, the operating hand of whom is denoted with numeral 7, is illustratively presented in FIG. 3. Following cutting a micro gripper 3 is employed (FIG. 4) for extracting each hair follicle 8 and the procedure continues with loading of the hair follicle within the frontal needle 20 of the hair implanting instrument as shown in FIG. 5 for sequentially approaching a selected locus at the recipient area of the scalp and pressing the press button assembly 15 to effect placement of the hair follicle 8 within the aforementioned preselected position as shown in the consecutive steps of FIG. 6. Following implanting and removal of the hair implanting instrument 2, FIG. 7 shows the newly implanted hair follicle 8 at an implanted condition in between a pair of previous hair implants 9 at the recipient area of the scalp, such newly implanted hair follicle 8 oriented at an angular inclination identical to that of the previous hair implants 9.

In accordance with a preferred embodiment of the invention, an initial test hair follicle extraction is performed in a small area of the donor region with a small number of hair follicles, so that the doctor may establish the correct depth and angle of hair growth in the donor area.

Following that, cutting continues and it is carried out in groups of hair follicles in a way that no more than 30% of the existing hair follicles are extracted in a single procedure. Care is taken so as to maintain a desired density of the donor region at a value exceeding 70 hair units/cm², in order to avoid certain areas of the scalp being made visible. The Technician must always pay attention to the symmetrical harvesting of the donor area. Upon reaching the desirable number of cut hair follicles, the first stage is completed; the donor area is cleaned, disinfected and covered with sterile gauzes.

In accordance with the invention it is preferred that the duration of the hair follicle cutting stage will not exceed a period of 4 hours. In accordance with this embodiment of the invention, the patient is thereafter allowed a proper short break and a light lunch. During the break, the design and hair distribution in the forthcoming implanting process are finalized by the medical practitioner and the client.

The patient is thereafter put in a seated position on a comfortable adjustable seat with adjustable neck support system with his neck bent forward 10-20°. A disinfecting step is repeated, this time at the recipient region of the scalp with an antiseptic solution (e.g. povidone iodine 10%) being applied for a period of about 1 min and then being removed with natural saline.

Following the extraction of the hair follicle, the medical practitioner consecutively examines hair follicles included in the plurality of extracted hair follicles and, according to the number of hairs incorporated in each particular hair follicle, places each hair follicle in the appropriate direction and proceeds with placement of the same at an appropriate position on the demarcated recipient region of the scalp. Each hair follicle 8 is individually loaded in the hair implanting device 2 and placed in the recipient area. The preferred mode of hair implanting of the invention without preexisting reception holes is recommended to start from the back towards the front and from the left towards the right of the scalp with a small angle towards the skin.

According to each specific recipient area, the medical practitioner appropriately adjusts the depth, the angle and the direction of implantation of hair follicles, in order to achieve a final natural and aesthetically optimum result when the hair is restored and grows back. Every hair follicle that is implanted is counted so that the total number of transplanted hair is known at the end of the procedure. Throughout the placement procedure, the recipient area is constantly being sprayed with normal saline and this is considered to essentially contribute towards ensuring the survival of hair follicles.

Upon completion of the hair placement procedure, the assistant technician checks all the implanted hair follicles and corrects any hair follicles placed too deep or too superficially or any hair follicles that might have accidentally been displaced.

Thereafter a check is made of the area to establish that it is thoroughly cleaned and both the recipient and the donor areas are sprayed with normal saline.

A form with the details of the procedure is finally filled out and post-operative quality control is carried out with the patient filling out the postoperative evaluation form. At the same time postoperative photos are taken. Thereafter the patient is provided with postoperative instructions for the rest of the day and the days to come and is discharged.

The treatment room is appropriately equipped for the application of the proposed technique with the aforementioned ergonomically designed patient and doctor adjustable seats and is provided with audio and video facilities, use of which is regulated by the medical practitioner in charge to the benefit of the patient's recreation and relaxation during the procedure. Short breaks are scheduled during the procedure in order for the staff and patient to rest and relax, whilst the medical practitioner in charge is responsible of ensuring that the overall procedure isn't extraordinarily prolonged.

The medical practitioner in charge does not leave the premises of the Clinic before the patient leaves the Clinic in good health. The operation room is finally properly cleaned and ready for the next procedure. Medical practitioners and technicians wear surgical caps and masks during the whole procedure and use clean gloves that are being changed frequently. To perform the procedure, the medical practitioner preferably uses magnifying glasses 10×.

1. Method of direct hair implantation consisting in sequentially harvesting single hair follicles from a donor region and directly implanting the same at a recipient region of the scalp, said method characterized in that it comprises in combination the steps of:
planning of the direct hair implantation procedure including selection of the hair line, the design and distribution of the hair follicles to be transplanted and employment of a surgical pen to demarcate the limits of the recipient area and hair distribution therein and for the demarcation of the limits of the donor region in the occipital and temporal areas of the scalp;
hair disinfection through washing with an antiseptic shampoo and hair cutting at an agreed, desired length;
appropriately positioning the patient seated on a properly adjustable seat with adjustable neck support in accordance with said hair transplanting planning step;
employment of a hair harvesting instrument with a sterile disposable punch of appropriately small diameter adapted to successively perform cutting of a single hair follicle when it is brought in contact with the root of the hair follicle and is manually rotated around the perimeter of the same;
employment of a micro gripper means for the extraction of the hair follicle cut during a hair harvesting operation and loading a forwardly projecting tubular needle of a hair implanting instrument with the extracted hair follicle;
employment of said hair implanting instrument for a selective placement of said hair follicle in the appropriate direction and at an appropriate position on the demarcated recipient region of the scalp, and repeating the single hair follicle harvesting, extraction and direct implantation operations until completion of the planned direct hair implantation procedure.

2. Method of direct hair implantation as claimed in claim 1, wherein an initial test hair follicle cutting and extraction operation is performed in a small area of the donor region with a small number of hair follicles, said initial test hair follicle extraction being examined with a scope of establishing the correct depth and angle of hair growth in the donor area.

3. Method of direct hair implantation as claimed in claim 1, wherein, following completion of the hair harvesting operation and obtaining of a desired, predetermined plurality of hair follicles, a cleaning and disinfecting step of the donor region and covering the same with sterile gauzes is performed.

4. Method of direct hair implantation as claimed in claim 1, wherein prior to initiation of the hair implantation operation, the method further comprises the steps of:
application of an antiseptic solution on the donor region and on the recipient region of the scalp for at least 1 min and of a sterile natural saline to remove the antiseptic solution after disinfection;
intradermal administration of a prepared solution of local anesthesia with a 30G needle in the donor region in a ring block manner, in accordance with a predetermined protocol of local anesthesia administration, wherein administration starts at the lower edge of the donor region and continues around the entire donor region to complete the ring block.

5. Method of direct hair implantation as claimed in claim 1, wherein said antiseptic solution applied on the donor and recipient region of the scalp with a scope of effecting disinfection thereof is a local anesthetic cream or gel that is applied in combination with ice.

6. Method of direct hair implantation as claimed in claim 1, wherein said appropriately small diameter of the sterile disposable punch used in performing hair follicle cutting in a hair harvesting operation lies within the range of 0.4 mm-0.8 mm.

7. Method of direct hair implantation as claimed in claim 1, wherein said placement of hair follicles in the appropriate direction and at an appropriate position on the demarcated recipient region of the scalp is carried out without any preexisting reception holes.

8. Method of direct hair implantation as claimed in claim 4, wherein said placement of hair follicles in the hair implantation operation is initiated from the back towards the front and from the left towards the right of the scalp with a small angle towards the skin.

9. Method of direct hair implantation as claimed in claim 1, comprising the steps of:
appropriately regulating and fine adjustment of the depth of intrusion of the forwardly projecting tubular needle of said hair implanting instrument into the scalp to provide for optimum performance of the hair implanting operation prior to initiation of the same;
withholding said extracted hair follicle within said forwardly projecting tubular needle, positioning said forwardly projecting tubular needle at a target recipient locus of the scalp and effecting a hair implanting operation of the hair follicle previously loaded within said forwardly projecting tubular needle through pressing a button means provided at the rear end of the hair implanting instrument thereby initiating a forward stroke of a push rod slidable within the interior of said forwardly projecting tubular needle to implant said hair follicle into said target recipient locus of the scalp, wherein a spring means axially disposed along the axis of the stroke of said push rod is being compressed and loaded during said forward stroke of said push rod and is subsequently unloaded thereby returning said forwardly projecting tubular needle at rest position for performing another hair follicle implanting operation.
